(19) United States
(12) Patent Application Publication
(10) Pub. No.: US 2009/0024008 A1
(43) Pub. Date: Jan. 22, 2009

(54) METHOD AND A DEVICE FOR SIMPLIFYING A DIAGNOSTIC ASSESSMENT OF A MECHANICALLY VENTILATED PATIENT

(75) Inventors: Josef Brunner, Chur (CH); Marc Wysocki, Chevreves (FR); Ivo Mettier, Felsberg (CH)

Correspondence Address: MORRISS OBRYANT COMPAGNI, P.C., 734 EAST 200 SOUTH, SALT LAKE CITY, UT, 84102, US

(73) Assignee: HAMILTON MEDICAL AG, Bonaduz (CH)

(21) Appl. No.: 12/162,778

(22) PCT Filed: Jan. 30, 2007
(86) PCT No.: PCT/CH2007/000041

§ 371 Date: Jul. 30, 2008

(30) Foreign Application Priority Data
Jan. 30, 2006 (CH) 147/06
Nov. 7, 2006 (CH) 1765/06

(51) Int. Cl. A61B 5/08; A61B 5/01

(52) U.S. Cl. 600/301

(57) ABSTRACT

The invention relates to a method for acquiring several changing values and representing the acquired values on a monitoring screen, as well as to a device with a screen in order, on this, to represent changing values acquired during ventilation of the patient. The device comprises means for acquiring at least three changing values of different origin, and means for representing the values, which permit the acquired values to be qualitatively represented on the screen together in a single graphic element. This graphic element has a pictorial representation of a lung shape. The invention is characterised in that the means for representing the values are designed such that a volume change of the ventilated lung which is detected within each breath, is represented in an animated manner by way of size change of the lung shape corresponding to this corresponding volume change.

### TECHNICAL FIELD OF THE INVENTION

The method relates to a method and to a device for acquiring several changing values which are be monitored during the ventilation of the patient, and representing the values on a screen.

The mechanical ventilation of patients, as the case may be, is necessary to keep the patient alive. This is for example the case with an anesthetic, or also with diseases or after an accident. A mechanical ventilation may however also damage the lungs of the patient. It is therefore the object of any ventilation treatment, to carry it out as briefly as possible and with the lowest possible intensity. Patient parameters and apparatus settings are to be used for estimating the necessity of ventilation, as well as the necessary intensity of the ventilation.

In order to adapt the ventilation where possible, to the requirements of the patient, a continuous, new assessment of the patient status and a likewise continuous, new assessment of the dependency of the patient on a mechanical ventilation is necessary. The dependency of the patient may be derived on the one hand from the present ventilation state, which may be recognized from the apparatus parameters set at the ventilator, and on the other hand from the patient status which is recognizable from the patient parameters.

### STATE OF THE ART

Conventional ventilators, anaesthetic apparatus or monitoring apparatus have a screen as a display, on which patient parameters measured by way of sensors, and/or the settings of a mechanical ventilation may be represented in real-time.

The representation of the patient parameters may mostly be selected between a representation in the form of numeric values or in the form of graphs. Some of such graphs in each case show an individual patient parameter in course of time, wherein the x-axis represents the time, and the measured or computed value of the parameter at the given point in time is plotted on the y-axis. The curve therefore indicates a reading of a parameter at a point in time of the past which is displayed in each case, and in particular in real-time. Several such graphs may be represented next to one another or over one another. Two curves may be represented simultaneously in a single graph. Some graphs show two patient parameters which are plotted against one another in so-called loops. The values of the one parameter are plotted on the x-axis, and the values of the other parameter on the y-axis. In the course of time, a point representing in each case both readings runs along a closed curve, a loop so to say. These loops are continuously updated by way of this. The point representing both readings in each case shows the current values of the measurements in real-time.

The known graphic representations of such readings are therefore two-dimensional curves which represent one patient parameter, or two patient parameters in the special shape of loops in real-time. A changing of the reading effects a shape change of the loop or the graph.

The representation of readings of the patient parameters and/or of values computed therefrom are used by the physician for estimating the patient status. The physician estimates how the patient is doing at that moment, by way of comparing these readings and graphs to normal values and tolerable deviations from these, which are known by him.

The apparatus setting with a mechanical ventilation may be read off from a series of numerically displayed setting values. The assessment of these values is necessary, in order to be able to estimate the dependency of the patient on the mechanic ventilation and to achieve a rapid withdrawal.

Such numeric and graphic representations may only be assessed spontaneously with great experience. The physicians must deal with the displayed values in an intensive manner, in order to be able to correctly assess the situation. Overlooking an individual parameter which does not support a withdrawal, which encourages a reduction of the intensity or which supports an increase in the intensity, may lead grave problems.

Research work is published in the “Journal of the American Medical Informatics Association” Volume 10, Number 4 of July/August 2003 with the title “The Employment of an Iterative Design Process to Develop a Pulmonaa Graphical Display”. In this, the development of a graphical display of the lung function for anaesthetics is specified as an example of an interactive fashioning method with an integrated usability test.

With the design specified as an example, the usability test resulted in an increase of the intuitive perception ability of the anatomical significance of the representation by 25%. The intuitive perception ability of the significance of the variable representation means was increased by 34%, and the precision of the diagnostic content was only reduced by 4%. The design creating these best test values finally displayed a graphic element which comprises the following picture elements: a graphically, very simplified lung with two lung lobes, a graphically very simplified trachea with two bronchial branches which reach into the lung lobes, and an inflating bellows is represented connected to the bronchi, whose height represents the tidal volume. A rectangle is represented left of the bellows, whose colour represents the oxygen content of the respiratory air, and a rectangle is represented on the right of the bellows, whose height represents the end-tidal CO₂ values. The respiration rate is above the bellows as a number.

Triangles may be imaged in the tracheae and in the bronchi which symbolise a respiratory resistance. The colour of the lung lobe symbolises the oxygen supply of the lungs. The compliance of the lungs is represented by two different outlines of the lung lobes: an outline representing a foam bubble represents an increased compliance, a gridded cage represents a reduced compliance.

The graphic representation is not animated. It is specified as an important aspect in the description, that the intuitive perception ability of the representation may change when the representation is animated with real-time data. Future studies however have yet to show whether the representation remains intuitively perceptible and usable when it is animated with the patient data.

### OBJECT OF THE INVENTION

It is therefore the object of the invention to simplify a diagnostic assessment of a patient undergoing ventilation, or to simplify an assessment of the parameters which are to be assessed with the ventilation of a patient. It is a further object of the invention to process and represent the parameters to be assessed. A further object of the invention lies in rendering the status of the patient or the dependency of the patient on the mechanical ventilation perceptible, in a manner which rapid and as intuitive as possible.

### Inventive Solution of the Object

According to the invention, this object is achieved by a method according to claim 1.

Such a method for detecting several, changing values of a different origin of a mechanically ventilated patient, and representation of the values on a monitoring screen, with the state of the art, has the feature that at least three of these values are acquired and qualitatively represented together in a single graphic element which encompasses a pictorial representation of the lung shape. It differs from this state of the art in that the current volume change of the ventilated lung is represented by way of the lung shape increasing and decreasing in size with each breath.

The animation of the lungs has the advantage, that the moving lung is directly understood as graphics representing the lungs of the patient. It indicates the rate, and may also qualitatively indicate the breath volume by way of the size change. Furthermore, one may directly recognise whether the lung does indeed move, i.e. whether the patient is indeed respirated, or whether for example an occlusion or a disconnection is present, in which case the lungs specifically advantageously have a fixed size.

The animation of the lung has the advantage compared to the animation of a bellows, that it is not the work of the ventilator which is rendered visible, but the effect of the ventilation.

If the lung shape is only able to experience size changes in one dimension, it is preferred for the lung shape in each case to be increased or decreased in size normally to a contour of the lung shape. This may be perceived in a very intuitive manner as a movement representing the respiratory movement. Such a size movement is perceived as an insufflation of the lungs and as an emptying of the lungs. It thus indicates a passive patient. The patient's own activity may be rendered visible in that the outline of the lung shape on the diaphragm side is changed in its fashion during the size change of the lung shape. The size change of the lung shape may therefore be effected in two ways, so that one may differentiate as to whether the patient actively breathes or is only ventilated. A trigger signal may be acquired in a manner known per se, for detecting whether the patient breaths actively.

The changing values are usefully measured on a patient being ventilated or possibly to be ventilated, by way of sensors. They may also be determined by calculation from such readings

Usefully the respiratory flow and/or the pressure conditions on the patient side are detected in a respiratory tube. With this, apart from the patient's own activity, one may also monitor as to whether a disconnection or occlusion is present. A constant value is allocated to the size of the lung shape, in the case that a disconnection or an occlusion is ascertained. The lung shape, in order to be able to intuitively differentiate the occlusion from the disconnection, is represented large in the case that an occlusion is ascertained, and in a small manner in the case that a disconnection is ascertained. One may envisage a normal lung shape size as a comparison value being superimposed on the moving or non-moving lung shape. This may e.g. be represented as a line or background area with the lung shape, with a size which is constant or a lung shape which increases and decreases in size. Such a line or area may likewise represent a comparison shape for the compliance of the lungs.

An airway shape is represented integrated into the lung shape, in the known manner. At least one changing value of a property of this airway shape, and which characterises the airway, is assigned to this airway shape. Preferably, the respiratory resistance is allocated to the width of the airway shape. Narrow airways are represented with a high respiratory resistance, and wider airways are represented with a low, i.e. normal breathing resistance. Apart from the width of the airway shape, the colour or the brightness level of the airway shape may also provide qualitative information on the respiratory resistance. Both properties may be combined with one another or used individually.

Furthermore, the graphic element may have a heart shape or a blood vessel shape, as also a lung muscle system in the form of a diaphragm below the diaphragm side of the lung shape. At least one changing value characterising the blood circulation is allocated to a characteristic of the heart shape or the blood vessel shape. Such values may be: blood pressure, represented by way of vessel width; oxygen saturation and CO₂ partial pressure, represented by two colours in part regions of the vessel- or heart shape; pulse, represented by a flashing or moving sign in the blood vessel shape or in the heart. A changing value characterising the participation of the patient in the respiratory work is allocated to a characteristic of this muscle system shape. Such a value may be a trigger signal, represented for example by way of a flashing-on of the muscle arch. The intensity of the participation may also be represented by way of the size of the muscle shape. Qualitative information on the activity of the lung muscle system may be provided in such a manner, which may be perceived in a rapid and intuitive manner. An activity of the lung muscle system is represented in several, for example in three, four, five or six graduations.

The change of the muscle system shape is effected usefully synchronously with the activity of the lung muscle system (strong-weak) and with the respiratory movement (inhalation, the arch becoming shallow; exhalation, arching of the muscle arch).

It is further desirable for the lung shape to provide qualitative information on the compliance. Advantageously, this is accomplished by a contour line which is thicker and/or unequally thick with a stiffer lung, and which is thinner with a more compliable lung. The contour fashion of the lung shape may also provide qualitative information on the stiffness or expansibility (compliance) of the lung. With a stiff lung, this is represented e.g. in an angular manner, with a compliant lung, in a bloated or wrinkly manner. A patterning of the lung area may likewise represent the compliance. The patterning advantageously has an asymmetry if the value for the compliance does not lie in the normal range. The patterning may for example represent a grid, whose grid fields are more angular with a hard compliance, and round with a soft compliance. The characteristics of outline shape, patterning and contour line may advantageously also together qualitatively represent the compliance and draw attention to this. The lung should furthermore also be represented in a more asymmetrical manner with an increasing deviation from normal values, in order to render the deviation more obvious.

The pulse may furthermore by displayed by the rhythm of a contour change of the heart- or vessel shape, or of a brightness change or a colour change of the heart- or vessel shape.

Lungs with airways and muscle system shape together usefully form a single graphic element which is to be read as a whole. The individual constituents have a fixed relation to one another. Usually the muscles and the lung lobes move in a synchronous manner. If the patent breathes against the rhythm of the ventilator it can be observed that the movements of the muscle shape and the lung shape are asynchronous according to the asynchrony between lung an muscle. The vessel/heart shape is incorporated into this graphic element. This increases the intuitive readability of the symbols, since these are perceived as human organs. The readability of the lung shape as a lung increases the readability of the muscle shape as a diaphragm. The readability of these picture components again makes it clear to the observer, that the vessel shape represents a vessel, and the heart shape a heart. The close relationship of one shape to the other within the graphic element has further the advantage, that asynchrony of the patients activity and the mechanical ventilation becomes visible.

Apart from the patient parameters which are rendered easily perceivable by way of this summary in the organ representations, the physician as a rule also needs the apparatus parameters, in order to be able to correctly estimate the situation. These are better shown in second graphics. For this reason, it is suggested to represent a second graphic element on the screen, which graphically represents three, preferably more than three, particularly preferably all of the following parameters:
the applied oxygen concentration “FiO₂”,
a positive endexpiratory pressure “PEEP”,
the inspiration pressure “Pᵢₙₛₚ” or in inspiratory mean pressure “Pᵢₘₑₐₙ”,
the ratio between controlled ventilation and spontaneous breathing “RRₜₒₜ/RRₛₚₒₙₜ, or the rapid, shallow breathing “RSB”,
the ratio of the theoretic minute volume (e.g. derived from IBW) to the current minute volume “MV”
the variability index and/or
an index for the breathing exertion of the patient (e.g. P0.1, which determines the breathing exertion on account of the vacuum generated by the patient, or an alternative index which determines the breathing exertion for example by way of the flow dynamics generated by the patient).

The physician, on account of these parameters or a selection of these parameters, may estimate how greatly the patient is dependent on the apparatus. These parameters too, should be able to be rendered perceivable as quickly as possible

For this purpose, these values are acquired and displayed in an analog or quasi-analog manner with a position of a division or of a positionally changing component on scales. Each scale is assigned to one of these parameters. The current value of each represented parameter is advantageously in each case shown on a range, said ranges in each case displaying poor values of the respective parameter at one end, and good values at a distance thereto, and these ranges are arranged in a manner such that the good values of all ranges are set in a relation to one another. This considerably simplifies the assessment of the displayed values.

The scale is advantageously divided into at least two regions. At least one region for good values and one for poor values, and as the case may be for middle values. These may differ by way of their colour, brightness or patterning. By way of the colour, brightness or patterning of one of these regions, one displays whether the displayed parameter value lies within or outside the region with good values. Advantageously, by way of the colour, brightness or patterning of a region of each range or of the background, it is displayed as to whether all displayed readings lie within the region with good values. If all values lie in the regions for good values, then one may consider disconnecting the patient from the ventilator. If at least one value is still not within this good region, then one may disregard to disconnect the patient from the ventilator.

Border line values of the regions may be fixed within the apparatus and therefore be unchangeable. In a preferred embodiment of the method, they may be pre-set, but they are definable and/or adjustable by the user. To define or adjust the regions, the user defines or adjusts the border line values between one region and an adjacent region using an interface.

A device according to the invention and corresponding to the method described above is designed as follows:

The device, in a known manner, is provided with a screen, in order on this, to represent detected changing values with the ventilation of a patient, and comprises means for detecting at least three changing values of different origin, and means for representing the values. These means permit the acquired values to be qualitatively represented on the screen together in a single graphic element. This graphic element in the known manner, has a pictorial representation of a lung shape.

This device-however differs from the state of the art initially mentioned, in that the means for representing the values are designed such that a volume change of the ventilated lung which is detected with each breath, is represented in an animated manner by way of the size change of the lung shape corresponding to this corresponding volume change. Accordingly, it is not only a breath volume which is pictorially represented, but the effect of the ventilation on the lung is pictorially represented in real-time. For this, sensors are usefully provided. In a preferred embodiment, a sensor for monitoring pressure and/or the flow of the respiration air is present on the patient side on a respiration tubing. With this, one may ascertain whether a disconnection or an occlusion is present, or whether the patient is effectively ventilated. An animated lung is represented on the monitor when the lungs of the patient expand with ventilation air and this air compressed into the lung is let out again. In the two mentioned cases, in which the ventilation does not take place, the lung shape is however shown on the screen with a constant size. This renders visible the effect on the patient, and not the task set in the ventilator.

Usefully, the constant size of the lung shape is large in the case that an occlusion is ascertained, and small in the case that a disconnection is ascertained. The surface change of the lung shape representing the breathing is advantageously in a relation with the measured tidal volume.

The represented lung shape advantageously also has a airway shape (trachea with bronchi). At least a changing value relating to the airways, in particular the resistance, is represented with this. A current ventilation resistance may also represented, or the deviation from a normal value may be emphasised, by the colour or the brightness level of the airway shape.

The lung shape may be divided up. An area division of the area of the lung shape advantageously represents a difference between an ideal lung volume and an actually ventilated lung volume. Such a division—spoken pictorially—is perceived as “water” in the lung and advantageously contains information as to which part of the lung does not participate in the breathing.

A current design of a contour line of the lung shape and/or a current configuration of an outline of the lung shape advantageously contain qualitative information on the stiffness or the compliance of the lung. This contour line and this outline are animated. They become larger and smaller with each breath. As already described, the counter line has a thickness corresponding to the compliance, and the outline configuration is more straight-lined and angular, the stiffer is the lung, and is rounder and more indented, the more compliant is the lung. A comparison value in the form of a taut, round line or such a background surface may be blended in, for comparison purposes. This represents an ideal compliance as a line following the outline of the ideal lung shape, or an area having the ideal lung shape. With this, one may furthermore represent an ideal breath volume.

Part of the graphic element is furthermore advantageously a muscle system shape. With this muscle shape usefully represented as a diaphragm represented below the lung, one may represent a changing value concerning the participation of the patient in the breathing work. Such a representation may be read in an intuitive manner. A thick muscle for example may be recognised as a large participation, and a thin one as a weak participation. The appearance of the muscle shape and the absence on the muscle shape are directly recognisable as a trigger signal of the patient, or as an absence of this.

A further constituent of the graphic element is advantageously a vessel shape or heart shape. This is suitable in order to represent a changing value concerning the blood circulation. A current blood pressure or any other haemodynamic index, in particular the current average blood pressure, may be represented by the current width of the vessel shape. A current oxygen saturation or any other index of oxygenation, and the current CO₂ partial pressure of the blood or any other index of ventilation may be represented by way of the current colour, at least of one region of the vessel shape and/or the heart shape.

The pulse may advantageously be indicated by way of a symbol which runs along a vessel shape or which flashes.

It may generally be stated that at least three different first graphic parameters corresponding to the origin, may be allocated to at least three values of a different origin, wherein first graphic parameters define different graphic basic characteristics of the element and of the background. The following are to be understood for example as graphic basic characteristics of the represented element and its background:

a position of the element on the screen or in relation to another element

a basic shape

a contour line

a colouring

a brightness

a patterning

a division or

a positionally changing constituent, etc.

These basic characteristics may in each case relate to the characteristic of the element and/or of the background.

The advantage of this association of the origin with the basic characteristic of the element or its background is that 3, 4, 5, 6 or more data each of a different origin are represented and are furthermore capable of being qualitatively assessed, in a single element. This is because an element has the above different basic characteristics, to which in each case a particular origin of the data may be allocated.

The qualitative representation of the reading or the apparatus setting is then effected on account of the value of the represented parameter. Second graphic parameters which define different conditions of the respective basic characteristic are specifically allocated to different values of the same origin. As conditions of the basic characteristics, one may mention:
size and design of the shape,
thickness of the contour line,
X defined colour from a colour scale,
specific brightness from a graduation of the brightness, in each case of the element area, of the background, of a contour line, of a moving component, of a part area, etc.,
design of a patterning on an existing area,
a contrast of the patterning,
positioning of the present division, definition of the division, width of the border region,
position of a present positionally changing component, etc.

If an assessment of one or more of the represented values with respect to a comparison value or a comparison value range is displayed, then this has the advantage that the assessment of the value may be effected in a quicker manner, and thus measures may be initiated more quickly, which is in the interest of the patient.

### BRIEF DESCRIPTION OF THE FIGURES

Examples of a representation achieved with the method are shown in the figures. The shapes and associations according to the description and the figures are exemplary and may be changed without departing from the invention defined in the claims. There are shown in:

FIG. 1 a screen of a ventilator with a representation of 14 values generated with the method according to the invention, and additional valuations of these values in the form of a schematic organ representation for the patient parameters, and a comparison scale bar for apparatus parameters which indicate the dependence of the patient on the ventilation.

FIG. 2 the same screen after opening an alarm window.

FIG. 3 a screen of a ventilator with the representation generated with the method according to the invention, and with an additional, conventional representation of two patient parameters.

FIG. 4 a screen section which represents the comparison scale bar with readings displayed therein, wherein several readings lie in a range which requires a continuation of the machine support of breathing.

FIG. 5 the screen section according to FIG. 4, wherein all readings lie in a range which could permit the termination of the machine support for breathing.

FIG. 6 a screen section which represents the organ representation with the readings displayed therein, wherein normal lung volume, a normal functional residual capacity, a poor compliance, a relatively high breathing resistance, a large muscle force and a high blood pressure are represented.

FIG. 7 the screen section according to FIG. 6, wherein however an unhealthy functional residual capacity, a normal compliance, a poor breathing resistance, a low muscle force and a low blood pressure are represented.

FIG. 8 the representation of a normal volume by way of the element size, with a normal compliance, represented by the element shape and the fine contour line.

FIG. 9 the representation of a low lung volume by way of the element size, in comparison to a comparison line or comparison area, with normal compliance.

FIG. 10 the active breathing of the patient by way of the lung shape change, in comparison to a comparison line or comparison area.

FIG. 11 the represented picture with absent readings (only comparison line or comparison area).

FIG. 12 a normal, functional residual capacity by way of division of the element area.

FIG. 13 an unhealthy, functional residual capacity by way of division of the element area.

FIG. 14 the represented picture with absent readings.

FIG. 15 a poor compliance (hard lung) by way of an asymmetrical, angular shape, and a contour line of the element whose thickness is variable.

FIG. 16 a normal compliance by way of a symmetrical outline shape with a taut, round leading of the lines.

FIG. 17 an excessive value for the compliance, represented by the asymmetrical, shrivelled bloated shape which partly reaches beyond the comparison area, and the very thin, almost absent contour line of the element.

FIG. 18 a normal breathing resistance by way of a complete filling of the element with a colour, and brightness of the colour.

FIG. 19 an increased breathing resistance by way of the distance between the coloured filling and the contour line of the element, and a brighter colour.

FIG. 20 an extremely high breathing resistance by way of a thin coloured area and its still brighter colour.

FIG. 21 the element with the absence of the reading for the breathing resistance.

FIG. 22 a low muscle activity of the patient by way of a lower thickness of the element.

FIG. 23 a high muscle activity of the patient by way of a large thickness of the element.

FIG. 24 a trigger signal without measurable muscle activity.

FIG. 25 a low blood pressure by way of the reduction of the diameter of the represented blood vessel, from the left to the right.

FIG. 26 a high blood pressure by way of the enlargement of the diameter of the represented blood vessel, from the left to the right.

FIG. 27 a normal blood pressure.

FIG. 28 the absence of a reading for the blood pressure.

FIG. 29 the pulse by way of a moved element on the standing picture for the blood vessel.

FIG. 30 the saturation of the blood with oxygen by way of the colour of the blood on the right side of the blood vessel.

### DETAILED DESCRIPTION OF THE INVENTION BY WAY OF THE EMBODIMENT EXAMPLES REPRESENTED IN THE FIGURES

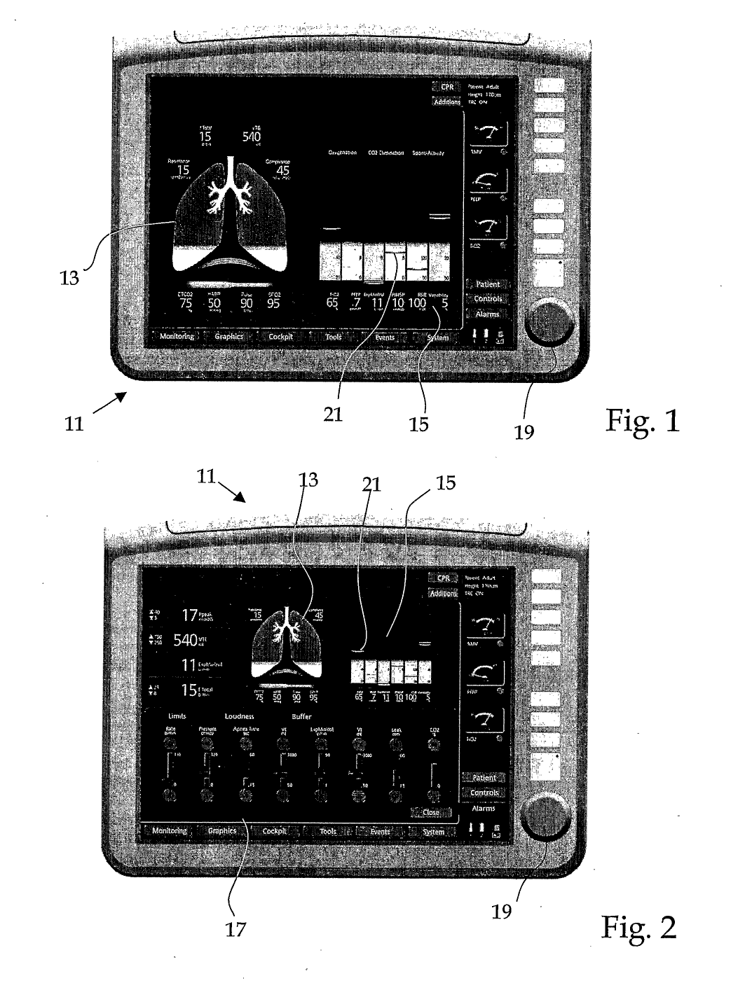

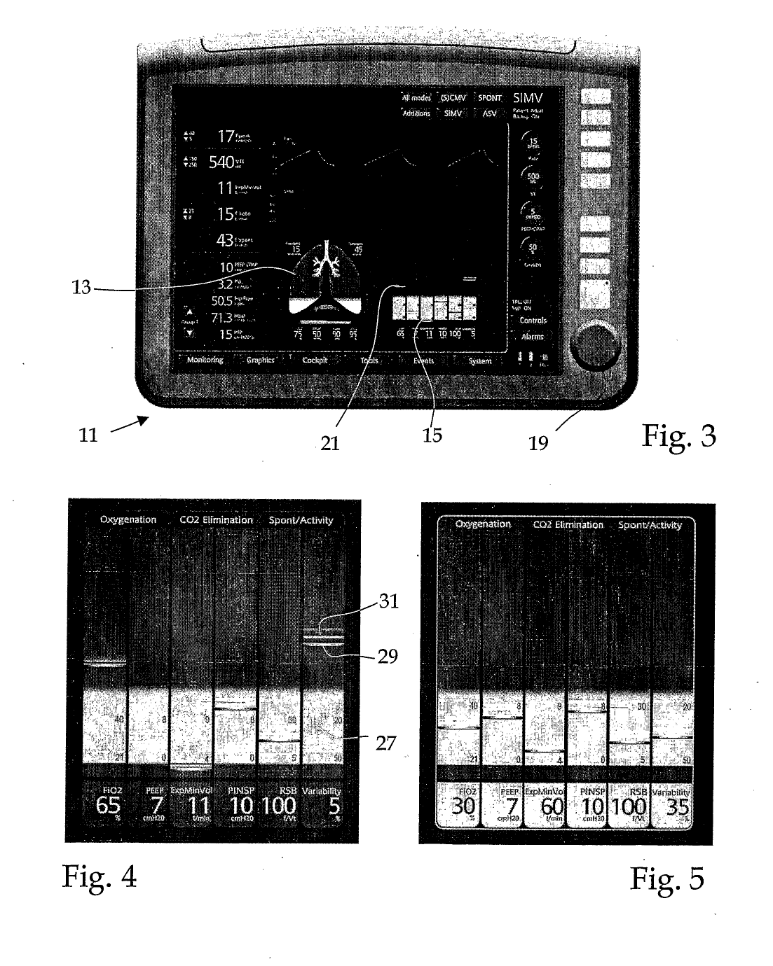

The screens 11 represented in the FIGS. 1 to 3 show representations generated with the method according to the invention. These representations comprise a first region 13 with patient parameters which contains an element reminiscent of an organ, and a region 15 with apparatus parameters which has a scale bar. The scales of this scale bar are different. Readings and apparatus settings are displayed on these scales with a positionally changing component 21. In FIG. 1, these two regions 13 and 15 practically take up the complete screen. In FIG. 3 they are squeezed together in the upper half of the screen, since a window is opened around the lower region. Such a window may be selected and opened with the selection button 19, whereupon the representation according to FIG. 1 changes into a representation according to FIG. 2. In FIG. 3, the representation of both regions mentioned above only takes up a part region of the screen, whilst an upper part region presents two graphs in a conventional manner, and a left screen region is filled with numeric values.

These different screen designs may be provided specific to the apparatus, or may be selectable with an apparatus.

The region 15 in which the apparatus parameters processed for an easier interpretation are represented, are shown in FIGS. 4 and 5. In the example, the following apparatus parameters are specified (from left to right):

FiO₂, the oxygen content of the respiration air, in percent;
PEEP, the positive end-expiratory pressure in cmH₂O;
ExpMinVol, the expiratory minute volume in litres per minute;
P_{INSP}, the inspiratory overpressure in cmH₂O;
RSB (rapid shallow breathing), an index for the shallowness or efficiency of the breathing in f/Vt (breathing frequency divided by tidal volume);
variability, an index which indicates the deviation from an absolutely regular breathing;
an index which indicates the breathing exertion. A known such index is called P0.1. This is measured on account of a vacuum in the ventilation system which is achieved over a short time period by way of the activity of the patient. An index comparable to P0.1 may be obtained by way of measuring dynamics of the flow of the ventilation gas created by the patient.

The first two scales provide information on the oxygen supply to the patient, which is why they are titled “oxygenation”, the middle two for ventilation, which is why they are titled “CO₂-elimination”, and the last two for the patient's own initiative, which is why they are titled “Spont/Activity”.

Each scale is directed in a vertical manner, and comprises a normal range 27 in which the values may be estimated as being favourable for the patient or for the course of the illness. This range is coloured and is highlighted with regard to brightness. In the example it is bright green, whilst the rest of the scale is dark grey and only highlighted very weakly from the black background of the screen. The exact reading is specified numerically below each scale. The same value is displayed in an analog manner with the help of a positionally changing indicator 29 within the scale.

In FIG. 4, the readings which are displayed on the scales for PEEP, P_{INSP} and RSB each lie within the normal range 27. For this reason, these normal ranges illuminate in a brighter green that the adjacent normal ranges of the scales, which indicate a range which is not to be classified as healthy or normal. In FIG. 5, all readings are within the normal ranges. For these reasons, all normal ranges illuminate in this brighter green. Furthermore, the background of the digital displays likewise illuminates in this green, in order to indicate that all scales indicate values which lie within the normal range 27.

Preferably, a single parameter with a reading within the normal range is not highlighted by colour, which is different to the shown variant. In contrast, the time duration since the occurrence of the reading in the normal range is displayed. In the case that all parameters have readings in the normal range, then all normal ranges together are highlighted by colour. A first blue indicates that not all readings yet lie within the normal range. A blue which is lighter compared to this, indicates that all readings lie within the normal range, and a withdrawal may be considered.

This apparatus parameter region 15 thus comprises six scales arranged next to one another, of which in each case two scales lying next to one another are grouped together, at least by a title or a common frame. The six scales, as shown in FIG. 5, are grouped together with a common frame. One scale, together with the numeric value detail, may be considered as an element. The following values are represented in this element: the origin of the readings accordingly defines the position of the scales with respect to the other scales. Each scale is an element with a background of a numerical display (changes from black to green) with a number thereon (changes from white to black), with a normal region highlighted by colour (changes in its brightness), with a positionally changeable indicator 29 (changes its position on the scale), with a trail 31 for the display of a tendency (depending on the tendency of the position change, is above or below the indicator 31, and may be shorter or longer depending on the speed of the value change (or position change).

The reading or setting value for the scale provided at a certain location is therefore represented in an analog and numerical manner (the agreement of this two display types is unfortunately not always given in the figures). The analog representation is effected via the position of the positionally changing indicator 29.

A value with a second origin is the fact as to whether this reading lies within or outside the normal region. This fact may also be represented by the brightness of the normal range 27, which has been highlighted by way of colour.

A value with a third origin is the fact as to whether all remaining values likewise lie within the respective normal range 27. This fact may be represented by way of the brightness and the colour of the background of the numbers of the numeric display, and the number in front of this background, and/or by way of the brightness or colour of all normal ranges.

A value with a fourth origin is a tendency of the development of the displayed value. This tendency may be displayed by a trail 31 of the positionally changeable component 29.

A value with the fifth origin is the speed of the change in this direction, said speed being able to be displayed by the length of the trail.

Therefore, with a glance of a single element, one may recognise which value e.g. for FiO₂ is set, whether this value lies in the normal range, in which direction the last setting correction was made, and, if FiO₂ lies within the normal range, whether the other readings- and setting values already likewise each lie in the normal region.

In the case of ExpMinVol, there are scale regions on both sides of the normal range 27, which display unfavourable values. These normal ranges 27 are at the end with the remaining scales. In contrast to the design represented in the figures, with individual or all scales, one may also add a transition region, differentiated by another colour, patterning or another brightness, between the normal range and the unfavourable ranges of the scales. The normal range 27 is merely blurred with respect to the unfavourable range in the represented embodiment.

The scales are compressed or extended in a manner such that the normal ranges have a unitary length. The scales on the other hand assume differently long ranges of the element length. A clear representation results by way of this, with which the approximation of an individual value to the normal range may be recognised in an excellent manner. Such an easy readability of the display may not be achieved with differently long normal ranges 27.

The normal ranges of the scales are arranged on a central axis, common to all. This would also increase the clarity of the display if the normal regions were to have different lengths

The readings which are represented in the scales of the elements and displayed therebelow in a numeric manner, are partly readings and partly setting values of the ventilator. The oxygen content of the ventilation gas FiO₂ may be reduced before the setting of the apparatus, or by way of a measurement by way of a sensor.

PEEP is usually likewise a setting value. ExpMinVol, the exhaled volume per minute, is computed from the flow measurement during the expiration phase, and averaged over several breaths.

Pinsp is mostly a setting value. This pressure may however also be monitored with a sensor. RSB, “rapid shallow breathing” is computed from measurements. It is based on a measurement of the rate and of the breathed volume.

The variability is computed on account of the differences of the temporal distances of the breaths, the differences in the length of the breaths and/or the volume differences with the breaths. A good variability indicates a larger independence of the patient from the ventilation, less variability however an larger dependency.

The region 13 with the representation of the patient parameters has a pictorial and qualitative display, which is accompanied by a numeric and quantitative display of a few readings. The numerical specification of the readings may also be made at a different location. It is evidently not a constituent of the graphic elements, which permits an easy readability and interpretation ability of the readings.

The representation of the patient parameters in the region 13 includes a schematic lung 31 with two lung lobes. The lung lobes are formed in a symmetrical manner with a healthy compliance. They both contain the same information. Furthermore, the representation includes a schematic airway tree 33, a schematic muscle system 35 and a schematic blood vessel 37. These part regions contain different amounts of information. They are described in detail further below.

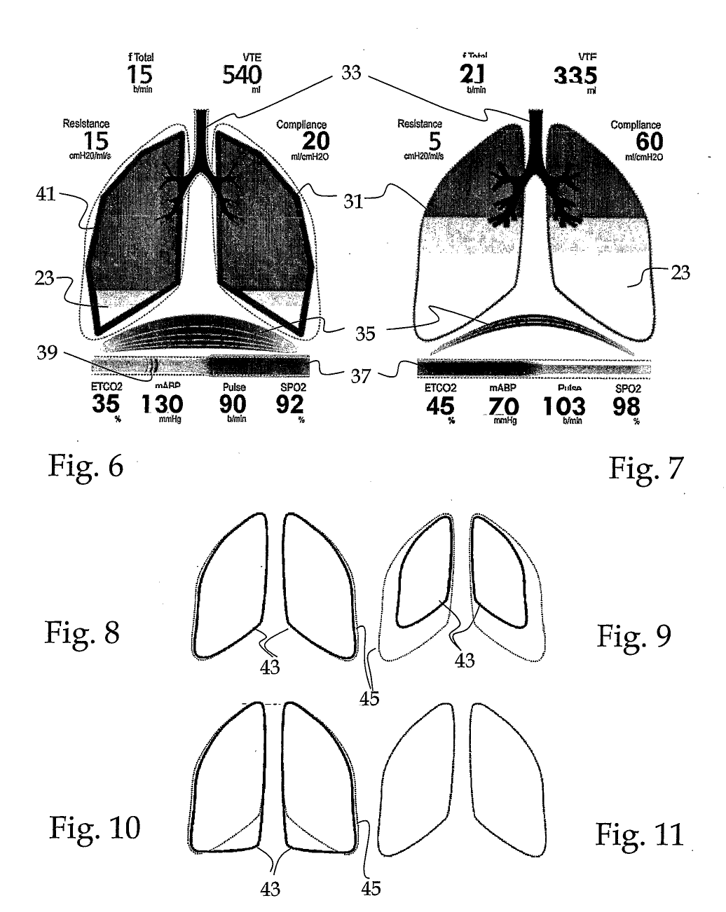

Two exemplary designs of the graphic element represented in the regions 13 are compared in FIGS. 6 and 7. Differences of the shaped formation of the element are immediately evident to the eye, whereas the number values around the graphic elements must firstly be read and then need to be interpreted.

In comparison to the conventional processing of readings and the setting values by way of graphs and numerical values, the advantage of the processing according to the invention becomes quite apparent by way of this representation of the respective data. Whilst the association of the number values and readings, as well as the graph forms is only possible via learned positional association, shape estimations or reading the lettering, such an association may be intuitively recognised with the representation of pictures which are reminiscent of organs. A difference 23 from an ideal total capacity of the lung or the muscle activity of the muscle 35 may for example be recognised immediately in the pictorial representation, whereas a number value would be significantly more difficult to interpret. The compliance for example is represented as a number value and as a graphic design of the element. The angular and thick contour line 41 in FIG. 6 evidently shows the hardness, whereas the round shapes being taut, and the fineness of the contour line in FIG. 7 may be recognised immediately by anyone as the healthy compliance of the lung. The association of the values 20 ml/cmH₂O or 60 ml/cmH₂O with a corresponding state of matters is however much more abstract even for experienced physicians, and thus may be recognised less quickly and reliably. It requires thought work, which thanks to the new type of processing of the data, may be invested to greater use in the consideration and the selection of therapeutic measures.

### The Lung 31:

The lung shape 31 consists of two mirror-imaged lung lobes. The lobes with the shown example are formed identically in a mirrored manner. They may however also be formed in an unequal manner, in order for example to make room for a heart between them.

The size of the lung lobes very coarsely indicates the lung volume. In FIGS. 8 and 10, the lung is filled with air, in FIG. 9, it is in the exhaled condition.

The lung lobes have an outline form. This outline form is designed differently, depending on the compliance of the lung. The shape of the lung is therefore allocated to the reading origin “compliance”. A poor compliance is shown in FIG. 15, a normal one in FIG. 16, and a compliance which is too large in FIG. 17. The compliance additionally to the representation via the outline shape, is also represented via the contour line 41. A low compliance may be recognised by a thick contour line which is straight-lined and angular. A high compliance, i.e. a flexible lung may be recognised by the thin contour line along a shape which is inflated with respect to the normal shape. Five or six graduations are provided: one for the flexible lung, one for the normal lung and three or four for differently stiff lungs. The compliance of the lung has a large band width and may be determined afresh with each breath.

With active breathing, the lung shape is different depending on the breathing condition of the lung. The lung is inhaled in an active manner in FIG. 10. The inner corners 43 of the lung lobes are pulled downwards, so that the lung lobes end at the bottom on a common straight line. On exhaling, the lung area decreases and the inner corners of the lung lobes return to the exhaled condition according to FIG. 9. In this condition, the inner corners 43 of the lung lobes are receded to the top, so that the lower edges of the lung lobes rise towards the middle.

The lung shape remains similar with an inactive patient. It then merely changes its area, in that the lung shape is spread from a centre (e.g. at the upper lung lobe tip).

E.g. 10 levels from FIG. 9 to FIG. 10 or from FIG. 9 to FIG. 8 are provided for the representation. The area of the lung increases synchronously with the volume curve on inhaling and reduces on exhaling, according to the current flow measurement

In FIG. 11, the lung shape is represented as is indicated with the absence of readings.

For this reason, the origin of the values of the activity/passivity of the patient is allocated to the breathing shape change of the lung. The size of the lung, or the breathing size change indicates the respiratory condition of the lung.

In each case, the contour 45 of a healthy, inhaled lung as a comparison value, is drawn in the FIGS. 8 to 11 and 15 to 17. In the representation of the readings, this comparison line serves for the assessment of the displayed lung size and the displayed compliance. The shapes and the size of the lung may be read off in an improved and unambiguous manner by way of this.

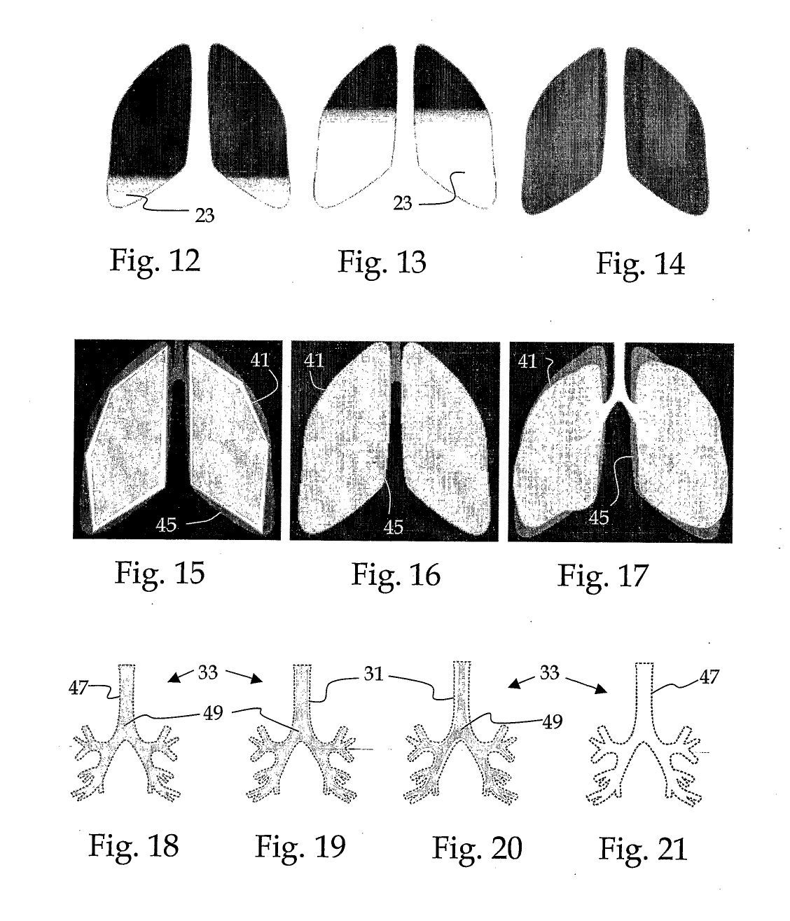

The lung lobes assume an area and have a coloured division of the area (FIGS. 12 and 13). The representation according to FIG. 14 indicates that no measurement of the functional residual capacity is present. If no measurement is available, the lung is grey and the area is undivided. If a measurement is available, the lung is divided into an upper blue, slightly patterned region, and into a lower, white region 23. The transition is shown in blurred manner. The division is designed in a different manner, depending on the size of the functional residual capacity (FRC) of the lung. The representation according to FIG. 12 still indicates a normal FRC, the representation according to FIG. 13, a poor FRC. The display is supported with new FRC readings after every approx. 60 minutes.

The colouring and the upper share of the lung are therefore allocated to a breathing volume measurement and to a measurement of the functional residual capacity, which are compared to an ideal lung volume (upper plus lower share) which is computed on account of the body size of the patient. The readings are mirrored in the position of the division defined by respective graphic parameters.

The parameters of tidal volume, functional residual capacity, compliance, lung size, active/passive breathing, and breathing condition of the lung, and thus also the breathing frequency may therefore be practically perceived at a single glance in the form of a temporal sequence of differently large shapes. The different parameters superimpose in the representation. The lung with a low compliance may likewise change its size and shape in the case that respective data are available, as with a lung of normal or high compliance. The movement of the lung lobes participate in the division of the lung into a difference 23 from an ideal total capacity and the given total capacity, and is independent of the compliance. The share of the functional residual capacity fills the lung lobes above the division, when these appear in expired representation (FIG. 9). In an inspired representation (FIG. 12 or 13) the upper share includes the present total volume of the lung. In a preferred embodiment, accordingly at least 4 or 5 different parameter are each represented in a qualitative manner.

### The Airway Tree 33:

A graphic element belonging to the lung is the airway tree 33. The airway tree 33 has a trachea which is represented in a simplified manner, with two bronchi and further branching. The basic shape of this airway tree already remains unchanged and is represented as a comparison value with the contour line 47. This airway tree graphically represents the breathing resistance, or resistance. The readings are represented in the form of a coloured airway tree 49 in the inside if this basic shape. With readings for a high respiratory resistance, the coloured airway tree 49 is designed in a thin-branched manner, whilst with a low respiratory resistance, the readings are represented as a thick-branched airway tree 49. Accordingly, the white line which fills the intermediate space between the inner, coloured airway tree 49 and the contour line 47, is thick with a high respiratory resistance, and is thin or absent with a low resistance. The brightness or colouring of the inner airway tree 49 is adapted to the respiratory resistance. With a high resistance, the colour is brighter and/or less intensive than with a low resistance. A healthy to pale pink is preferred as a colour. Such adaptations may however also be effected in a manner different to that described. Thus the airway tree may be represented in an alarm colour with a high resistance.

The measurement of the breathing resistance may be made with each breath. It generates data with a band width of 0 to 50 cmH₂O/ml/s. Three levels are provided for the representation of the resistance. No coloured airway tree 49 but only the contour of the basic shape is represented with the absence of readings.

### The Diaphragm 35:

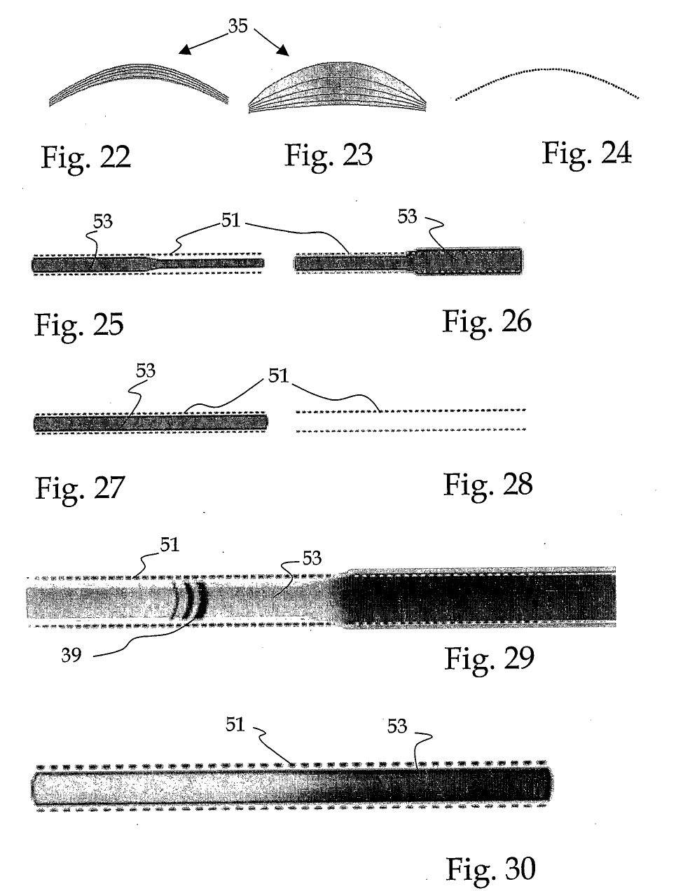

The muscle 35 represented as a stylised diaphragm may likewise be counted as belonging to the lung. The dimension of the muscle 35 indicates how greatly the patient breathes himself. A strong muscle according to FIG. 23 shows a large participation of the patient in the breathing work. A weak muscle according to FIG. 22 shows a poor participation of the patient in the breathing work. The absence of the muscle indicates that the patient does not participate in the breathing work, or that no readings are present. A muscle line according to FIG. 24 indicates that a trigger signal is present. This muscle line illuminates with a trigger signal and fades again thereafter.

The muscle 35 changes its shape with patient activity, but remains equally strong. It extends on breathing in, and curves on breathing out. The shape of the lung lobes moves parallel thereto, as described. In each case, 10 levels between the inhaled and exhaled lung are provided for both muscle strengths for the representation of the movement.

The blood vessel 37:

The representation of values with regard to the blood, specifically the mean blood pressure, the pulse and the saturation of the blood with oxygen, for example the arterial CO₂-partial-pressure is graphically independent of the lung, airways and muscle system. These values are represented by a stylised blood vessel 37. A heart may also be represented instead of a blood artery 37.

The blood vessel 27 has a left and a right side, which in the representations according to FIG. 25 to 30 are only separated by the brightness of the areas and/or by the shape. In reality, a coloured difference is also present, which however is not pictorially represented in this document. For the case that no readings and no pulse are present, only the contour line 51 of the blood vessel according to FIG. 28 is displayed. If no reading is present for the oxygen saturation of the blood, but only one for the blood pressure, the area is represented grey (FIG. 25-27), in order to be able to indicate the blood pressure. If only one pulse is displayed, the blood vessel is filled in grey, as is represented in FIG. 27.

The gas exchange is effected schematically in the middle of the blood vessel. This middle may be marked with a shape, e.g. with a circle, a point or a lung shape. A positionally changeable component (FIG. 29) in the form of a pressure wave symbol runs with the rhythm of the pulse from left to right. The blood “runs” in the same direction. This means: the blood is represented before the gas exchange left of the middle. There, its colour, as the case may be, can indicate the arterial CO₂-partial-pressure. The blood is represented after the gas exchange to the right of the middle. There, the colour indicates the oxygen saturation of the arterial pressure. The reading which indicates the CO₂-partial-pressure, is qualitatively shown in two graduations of the colour blue. The reading which indicates the oxygen saturation, is qualitatively shown in two graduations of the colour red, specifically pink and magenta. A high oxygen saturation is represented by a full magenta of the right blood vessel side in the FIGS. 29 and 30.

A haemodynamic evaluation is represented by the width of the coloured or grey filling area 53. It is suggested to leave the width of the filling area 53 constant in the left part of the blood vessel. The width of the filling area 53 is varied on the right side of the blood vessel, in order to qualitatively display the average arterial blood pressure. A wide formation of the right-side filling area 53 beyond the width of the filling area 53 on the left side, and a comparison contour 51 of the blood vessel, indicates a high blood pressure reading (FIG. 26). A narrow, right filling area 53 however indicates a lower reading for the blood pressure (FIG. 25). A normal blood pressure is displayed with a constantly wide filling area 53 (FIG. 27).

The blood pressure may also be indicated in a manner which differs from the above cited manner. It is e.g. possible, according to the blood pressure, to narrow or widen the shape of the blood vessel as a whole from the left to right, to inflate or contract a region. The blood pressure could be represented over the whole length of the blood vessel by the width of the filling area 53, a thickness of a vessel contour line, or another changing characteristic of the representation.

One differentiates between only four representations: high blood pressure, normal blood pressure, low blood pressure, no measurement. The representation is concluded in each case after 5 to 60 minutes on the basis of new readings. It represents the average arterial blood pressure and has a band width of 0 to 250 mmHg.

If no measurement of the blood pressure is present, the saturation of the blood or the respective arterial partial pressure may for example be represented by way of a coloured point on the left and right side (not shown in the figures).

For this reason, the following three parameters are represented in a qualitative manner with the help of the blood vessel 37, e.g. by way of the shape, the colour and a moving component: blood pressure; oxygen saturation, pulse. Furthermore, one may possibly represent the CO₂-partial-pressure.

The values for the compliance and the resistance are determined via a measurement of flow and pressure on breathing in and out, and displayed numerically as well as graphically. These measurements are for example made with a two-way flow sensor which is close to the patient, and the pressure sensors belonging to the ventilator, and converted in the known manner, in order to obtain values for the compliance and the resistance. The determined values, then according to their magnitude, are assigned to one of six (compliance) or one of three (resistance) different graphic parameter values.

Values for the number of breaths per minute are taken from the ventilator and, as the case may be, are combined with trigger signals activated by the patient. For example, a time measurement is taken over eight breaths, and converted into a number of breaths per minute. This computed value is rounded, and numerically displayed in whole numbers. The representation of the lung movement however shows the breathing movement synchronously with a volume which is determined with the flow measurement mentioned above. Individual short breaths as well as pauses in the breathing movement are represented as such.

The expiratory tidal volume VTE is determined by way of integration of a flow measurement over the expiration phase. This measurement and computation value forms the basis for a numerical display of a volume, but also for the graphic, pictorial representation of the lung size. One applies the body size for computing the pictorially represented lung size. The lung size is composed of the tidal volume and the functional residual capacity (upper share), as well as a difference to an ideal lung size. The tidal volume is measured with each breath. The functional residual capacity is determined by a manoeuvre, in which specifically the oxygen concentration in the respiration air is changed in a defined scope, and the effect on the CO₂-content and the O₂-content of the exhaled air is measured. Then, on the basis of these readings and setting values, one may draw conclusions on the functional residual capacity by way of calculation.

The upper share of the total area of the lung representation is then computed from the tidal volume and the functional residual capacity, and the respective area shares from the difference to an ideal lung volume. On breathing in and out, the volume of the respiration gas is measured and changed according to the size of the lung representation. With this adaptation of the lung size on breathing in and out, in each case one applies the most recent evaluation of the tidal volume and of the residual capacity, and the currently measured respiratory volume is added or subtracted. The maximum lung size is independent on the actual, ideal lung size in comparison to the IBW. The change of the lung size there represents a percentage change.

The CO₂-content of the expired breathing air is measured with a suitable sensor. The CO₂-partial-pressure of the blood may also be measured transcutaneously, as the case may be, also invasively. This reading is displayed in a numeric manner, but is also used for determining the functional residual capacity with the help of the described manoeuvre.

The blood pressure is measured sporadically with a sleeve, or invasively with a catheter. The average arterial blood pressure mABP is determined from the readings. This reading is numerically displayed in an exact manner, but only allocated to three graphic parameter values, specifically low, normal and high.

The oxygen saturation and the pulse are measured with a pulse oximeter. Invasive or transcutaneous measurements are however also possible. Instead of the saturation, one may also measure a partial pressure. The reading for the oxygen content of the blood is displayed in a numeric manner, but only allocated to two graphic parameters, specifically poor and good. The reading for the pulse is converted to a pulse rate per minute and displayed numerically. Each pulse stroke however causes the pressure wave symbol 39 to begin to run from the left to the right over the length of the blood vessel 37. The speed of the symbol 39 may be changeable, and increase and reduced according to the pulse rate.

1. A method for acquiring several, changing values of a different origin of a mechanically ventilated patient and representing the values on a monitoring screen, comprising:
acquiring at least three values, qualitatively representing together in a single graphic element a pictorial representation of the lung shape, and
representing the current volume change of the ventilated lung by way of the lung shape increasing and decreasing in size within each breath.

2. The method according to claim 1, further comprising increasing or decreasing the lung shape in each case in size essentially normally to a contour of the lung shape.

3. The method according to claim 1, further comprising changing in its fashion, during the size change of the lung shape, the outline of the lung shape on its diaphragm side, in the case that the patient actively breathes.

4. The method according to claim 1, further comprising measuring the changing values by way of sensors on a patient being ventilated or possibly to be ventilated, or are determined from such readings by calculation.

5. The method according to claim 4, further comprising acquiring the respiratory flow and/or the pressure conditions in order to monitor whether a disconnection or an occlusion occurs, and assigning a constant value to the size of the lung shape, in the case that a disconnection or an occlusion is ascertained.

6. The method according to claim 5, further comprising representing the lung shape in a large manner in the case that an occlusion is ascertained, and in a smaller manner in the case that a disconnection is ascertained.

7. The method according to claim 1, further comprising representing an airway shape integrated into the lung shape, and assigning at least one changing value characterising the airway to a characteristic of the airway shape.

8. The method according to claim 7, further comprising making qualitative information on the ventilation resistance by way of the width of the airway shape and/or by way of the color or the brightness level of the airway shape.

9. The method according to claim 1, further comprising monitoring the activity of the lung muscle system, the represented graphic element having a muscle system shape, which represents a lung muscle system, in particular a diaphragm, and assigning a changing value characterizing the participation of the patient with the breathing work to a characteristic of the muscle shape.

10. The method according to claim 9, further comprising representing an activity of the lung muscle system in several levels.

11. The method according to claim 9, further comprising effecting a changing of the muscle system shape synchronously with the activity of the lung muscle system.

12. The method according to claim 1, further comprising including in the graphic element a heart-shape and/or a blood-vessel-shape, and assigning at least one changing value characterizing the blood circulation to a characteristic of the heart shape and/or the blood-vessel-shape.

13. The method according to claim 12, further comprising providing qualitative information on the haemodynamic status, in particular the average blood pressure, by way of the width of at least a part region of the vessel shape.

14. The method according to claim 12, further comprising providing qualitative information on the oxygen saturation of the blood by way of the color of at least a region of the vessel shape and/or the heart shape.

15. The method according to claim 12, further comprising providing qualitative information on the carbon dioxide partial pressure of the blood is way of the color of at least one a region of the vessel shape and/or of the heart shape.

16. The method according to claim 12, further comprising displaying the pulse by way of the rhythm of a contour change of the heart shape or a brightness change or a color change within the heart shape.

17. The method according to claim 12, further comprising displaying the pulse by a symbol running along the vessel shape or by a symbol pulsating in or at the vessel shape.

18. The method according to claim 12, wherein the colour or brightness level of the heart shape or of the vessel shape provides qualitative information on the oxygen supply of the patient.

19. The method according to claim 1, further comprising representing a second graphic element on the screen, which graphically represents at least three of the following parameters:
an applied oxygen concentration “FiO₂”,
a positive end-expiratory pressure “PEEP”,
an inspiration pressure “Pᵢₙₛₚ” or in inspiratory mean pressure “Pᵢₘₑₐₙ”,
a ratio between controlled ventilation and spontaneous breathing “RRₜₒₜ/RRₛₚₒₙₜ” or the rapid shallow breathing “RSB”,
a ratio of the theoretic minute volume (e.g. derived from DBW) to the current minute volume “MV”
the variability index
an index for the respiratory exertion of the patient.

20. The method according to claim 19, further comprising displaying the parameter values with a position of a division or of a positionally changing component, in an analog or quasi-analog manner on scales allocated to the parameters.

21. The method according to claim 19, further comprising representing the current value of each represented parameter in each case on a range, said ranges in each case displaying poor values of the respective parameter at one end, and good values at a distance thereto, and these ranges are arranged in a manner such that the good values of all ranges are graphically set in a relation to one another.

22. The method according to claim 19, further comprising representing at least one region with good values, one with poor values, and, as the case may, with middle ones are red, and are differentiated by way of their color, their brightness and/or by way of their patterning, wherein by way of the color, brightness or patterning of a region of each range or of the background, it is displayed as to whether all displayed readings lie within the region with good values.

23. The method according to claim 22, further comprising defining or adjusting the regions by the user defining or adjusting border line values between one region and an adjacent region.

24. The method according to claim 1, further comprising measuring or computing the stiffness or compliance of the lung on the bases of readings and that a current formation of a contour line and/or a current fashion of an outline of the lung shape is adapted according to the stiffness or compliance of the lung.

25. A device with a screen to represent acquired, changing values with the ventilation of a patient, comprising:
means for acquiring at least three changing values of different origin, and
means for representing the values, which permit the acquired values to be qualitatively represented together on the screen in a single graphic element;
said graphic element including a pictorial representation of a lung shape,
the means for representing the values are designed such that a volume change of the ventilated lung which is acquired with each breath, is represented in an animated manner by way of a size change of the lung shape corresponding to the volume change.

26. The device according to claim 25, further comprising a sensor for monitoring pressure and/or the flow of ventilation air in order to be able to ascertain a disconnection or an occlusion, and that in both cases the lung shape is represented with a constant size on the screen.

27. The device according to claim 26, wherein the constant size of the lung shape is large in the case that an occlusion is ascertained, and the constant size of the lung shape is small in the case that a disconnection is ascertained.

28. The device according to claim 25, wherein the area change of the lung shape representing the breathing is in relation to a measured tidal volume.

29. The device according to claim 25, wherein an area division of the area of the lung shape is present, which represents a difference between an ideal lung volume and a ventilated lung volume.

30. The device according to claim 25, wherein a current formation of a contour line and/or a current fashion of an outline of the lung shape has qualitative information of the stiffness or compliance of the lung.

31. The device according to claim 25, wherein an ideal lung volume, and/or an ideal compliance is represented as a line following the outline of the ideal lung shape, or an area with the ideal lung shape which is stepped with regard to color or brightness.

32. The device according to claim 25, wherein the represented lung shape also includes an airway shape and at least one changing value concerning the airways is represented with the airway shape.

33. The device according to claim 32, wherein a current ventilation resistance is represented by way of the current width of an airway shape and/or by the color or brightness level of the airway shape.

34. The device according to claim 25, wherein the graphic element also includes a muscle system shape which represents a muscle system, in particular a diaphragm, and at least one changing value concerning the participation of the patient in the breathing work is represented with the muscle shape.

35. The device according to claim 25, wherein the graphic element also includes a vessel shape, and at least one changing value concerning the blood circulation is represented with the vessel shape.

36. The device according to claim 35, wherein a current blood pressure, in particular the current average blood pressure is represented by the current width of a vessel shape.

37. The device according to claim 35, wherein a current oxygen saturation of the blood is represented by way of the current color at least of one region of a vessel shape and/or a heart shape.

38. The device according to claim 35, wherein a symbol of the pulse which runs along a vessel shape or which flashes is displayed.

39. The device according to claim 25, wherein a second graphic element is represented on the screen, which graphically represents at least three, of the following parameters:
an applied oxygen concentration “FiO₂”,
a positive endexpiratory pressure “PEEP”,
an inspiration pressure “Pᵢₙₛₚ” or in inspiratory mean pressure “Pᵢₘₑₐₙ”,
a ratio between controlled ventilation and spontaneous breathing “RRₜₒₜ/RRₛₚₒₙₜ”, or a rapid shallow breathing “RSB”,
a ratio of the theoretic minute volume (e.g. derived from IBW) to a current minute volume “MV”
a variability index
an index for the breathing exertion of the patient.

40. The device according to claim 39, wherein the parameter values are displayed in an analog or quasi-analog manner with a position of a division or of a positionally changing component, on scales allocated to the parameters.

41. The device according to claim 39, wherein the current value of each represented parameter is represented in each case on a range, said ranges in each case displaying poor values of the respective parameter at one end, and good values at a distance thereto, and these ranges are arranged in a manner such that the good values of all ranges are graphically put in a relation to one another.

42. The device according to claim 41, further comprising an interface for entering border line values between one region and an adjacent region.

43. The device according to claim 39, wherein at least one region with good values, a region with poor values, and as the case may, a region with middle values are represented, and are different by way of their color, their brightness and/or by way of their patterning, wherein by way of the color, brightness or patterning of a region of each range and/or of the background, it is displayed as to whether all displayed readings lie within their region with good values.
